(19) United States
(12) Patent Application Publication
(10) Pub. No.: US 2013/0216634 A1
(43) Pub. Date: Aug. 22, 2013

(54) Herbal Supplement Prepared From Pelargonium Citronellum

(71) Applicants: James Akrong, Oakville (CA); Shawn Shirazi, Oakville (CA); Jason Peters, Oakville (CA); John Doherty, Oakville (CA)

(72) Inventors: James Akrong, Oakville (CA); Shawn Shirazi, Oakville (CA); Jason Peters, Oakville (CA); John Doherty, Oakville (CA)

(21) Appl. No.: 13/795,411

(22) Filed: Mar. 12, 2013

Related U.S. Application Data
(63) Continuation-in-part of application No. 13/038,793, filed on Mar. 2, 2011, now abandoned.

(51) Int. Cl. A61K 31/131; A61K 31/197; A61K 31/198; A61K 36/899; A61K 36/24; A61K 31/137; A61K 31/133; A61K 36/68; A61K 31/522; A61K 36/41

(52) U.S. Cl. CPC A61K 31/131; A61K 31/522; A61K 31/197; A61K 31/198; A61K 36/41; A61K 36/24; A61K 31/137; A61K 31/133; A61K 36/68; A61K 36/899
USPC 424/738; 514/663; 514/263.34; 514/564; 514/565; 424/725; 424/750; 264/12

(57) ABSTRACT

An extraction method for extracts of Pelargonium citronellum with improved methylhexaneamine content is provided. The method involves separating the oil phase from the aqueous phase; concentrating the aqueous phase; purifying the oil phase; and recombining the resulting material. Additionally, extracts of Pelargonium citronellum prepared by the extraction method are provided. The extracts are useful in compositions, for example as dietary supplements, and for appetite suppression.

### FIELD OF THE INVENTION

This is a continuation of U.S. application Ser. No. 13,113,475, filed on May 23, 2011, pending, which is in turn a continuation in part of U.S. application Ser. No. 13/038,793, filed Mar. 2, 2011, presently abandoned.

The present invention relates to herbal supplements and, more particularly, to herbal supplements derived from particular species of Geranium or Pelargonium according to a specific extraction method. In certain embodiments, the present invention relates to methods for suppressing appetite in a subject, comprising administering a composition comprising an extract of Geranium or Pelargonium, wherein the extract contains at least 1% or more of methylhexaneamine.

### BACKGROUND OF THE INVENTION

Geranium species are common throughout temperate regions and are used in many different parts of the world in traditional systems of medicine. ‘Geranium oil’ is also widely used; however this normally refers to the essential oil distilled from Pelargonium species rather than Geranium. In general, most species are used for similar disorders, although there are some different local indications for each. Prior to the instant invention, Geraniums used medicinally usually contain high levels of tannins, which are responsible for the traditional use as haemostatics and astringents. They are used internally for haemorrhage and diarrhoea, and externally for wounds, grazes, sores and fissures. More recently various tannin-containing drugs, including Geranium species, have been used as anti-infective agents particularly for viral diseases and antioxidant activity, which would be expected from the content of polyphenolic compounds.

Numerous parts of the world have produced Geranium oil in the past, but recently this has proven to be non-economical for large-scale production. Small amounts of Geranium oilis are produced in India, Morocco and Algeria, largely for domestic consumption. In Kenya, Geranium oil is known as Mawah oil, and is most often isolated from P. fischeri.

Geranium concrete and absolute are made in small amounts for certain perfumes and are produced mainly in Egypt. The concrete, extracted with petroleum spirits or hexane is dark green or brownish-green with a foliage-like odour and great tenacity. The Geranium absolute, made from the concrete by dissolving in absolute alcohol and then chilling to precipitate the insoluble components, followed by evaporation of the solvent, is also greenish with a somewhat leaf-earthy and powerful odour. Terpeneless Geranium oil can be produced from the Geranium oil and the absolutes by vacuum distillation; this makes the oil more soluble in diluted alcohol and is useful in foods as well as cosmetics.

One of the main products of Geranium oil and absolutes in the past was rhodinol, which is composed mainly of the citronellol fraction. This was used extensively in the ‘poor-man's’ rose perfumes and cosmetics, including soaps, creams, etc. Nowadays, rhodinol is produced synthetically, as the price of geranium has soared.

The major constituents of Geranium oil are generally rhodinol, geraniol, citronellol and their esters, which give the oil its aroma and commercial value. Geranium oil therefore can be easily concocted from cheaper essential oils and adjusted to the recommended ISO (International Organization for Standardization) standards. The antimicrobial activity of such essential oils is much greater than that of some authentic oils but has a similar pharmacological effect on smooth muscle (spasmolytic) and the actual odour can be even more appreciated by perfumers than the real essential oil. The essential oil composition of this Geranium oil differs completely from that of true G. robertianum oil or that of G. maccrorhizum. Thus, while adulterated geranium oil synthesized from recipes may be satisfactory for many uses, it will not provide the appetite-suppressing benefits provided by extracts of the present invention.

Adulteration of Geranium oil is perhaps encouraged by the ISO requirements themselves and the comparatively low price of synthetics. The yield of Geranium oil is less than 0.3 per cent, and is usually 0.2 per cent. Adulteration of all essential oils occurs to a considerable extent with diluents like propylene glycol, triacetin, triethyl citrate or benzyl alcohol, ethyl alcohol and in the case of aromatherapy oils with fixed oils like almond oil, which are added in excessive amounts. Adulteration also implies giving the wrong source on the labelling e.g. Bourbon, if it came from another country or was synthetic.

Like Geranium oil itself, Rhodinol ex Geranium is often adulterated with synthetic rhodinol, fractions of citronella or palmarosa oils and synthetic components. G. macrorrhizum (Zdravetz oil), produced almost solely in Bulgaria, has been used for adulterating Geranium oil. The essential oil composition of this Geranium oil differs completely from that of true G. robertianum oil or that of commercial Geranium oil from Pelargonium cultivars. G. robertianum contains mainly terpinene, linalool, terpineol, and an assortment of monoterpenes in contrast to commercial Geranium oil with citronellol and geraniol as its main components.

The percentage of linalool in G. robertianum is considerably higher than that in commercial Geranium oil, which is about 3-10 per cent, which was based on the actual analytical data of over 40 commercial Geranium oils from different geographical sources (as on labels) bought from many different commercial outlets). Adulteration with G. robertianum oil would therefore be easily detected using conventional gas chromatography as well as simply by its smell.

The apparent geographical source had on the whole no correlation with the chemical composition of commercial Geranium oil except for the presence or absence of the relevant sesquiterpene: i.e. 10-epi-g-eudesmol in Egyptian oils (3-7 per cent) and guaia-6,9-diene (1-7 per cent) in the Bourbon and China oils; a Moroccan oil contained both these sesquiterpenes. The proportion of the main components i.e. citronellol, geraniol, linalool, iso-menthone, citronellyl formate and geranyl formate was not consistent for any geographical source. The bioactivity, as determined by the action of the oils against 25 different bacterial species, 20 different Listeria monocytogenes cultivars, three different fungi and also their anti-oxidant action was not correlated with either the geographical source of the Geranium oil specimens or their chemical composition. The activity of the main components, citronellol and geraniol, was assessed against all the bioactivity parameters either singly or in combination, in the percentages listed by the ISO for different Geranium oils. The bioactivity was very potent for both the components, and the mixtures. However, a sample of Australian oil extracted using field distillation and obtained directly from its source, was comparatively inactive, suggesting possible adulteration of commercial oils with synthetic components.

The effects of different samples of Geranium oil were also investigated pharmacologically using guinea-pig ileum in vitro. There was again a variation in the bioactivity as shown by the relaxation produced in the smooth muscle. There was insufficient variation to warrant this to be a sensitive method for Geranium, but other work using enantiomers have indicated that there is scope for seeing differences in activity due to individual enantiomers which react differently in different tissues.

Methylhexaneamine (also known as methylhexanamine or dimethylamylamine) has previously been demonstrated to comprise no more than 0.66-1% of Geranium oil. The yield of Geranium oil itself is generally less than 0.3%, thus dimethylamylamine represents an insignificant fraction of Geranium species which has been uneconomical to extract considering the ease with which it can be synthesized.

Methylhexaneamine has become a popular dietary supplement, sold as a “pre-workout” product with a variety of added ingredients usually including caffeine. Due to the uncertain regulatory status of methylhexaneamine in the United States and elsewhere, natural sources of this dietary ingredient are highly sought after. As with Geranium oils used in perfumes and food, adulteration (in this case with synthetic methylhexaneamine) is perhaps the norm. The invention herein disclosed is the first example of a natural Geranium extract economically providing a source of methylhexanamine.

US2010/0041622 describes 2-amino alkanes, including methylhexaneamine, and their activity as vasoconstrictors, resulting in a number of potential physiological effects. No source of the 2-amino alkanes is identified.

Natural products, including simple alcohol extracts of herbs are highly sought after and so economical sources of such natural extractives are desirable. As described herein, extracts of Geranium providing at least 1 mg, and preferably at least 3 mg, of methylhexaneamine per serving are desired in order to suppress the appetite of a person desirous of such effect. Practical limits on a pill form of supplement require that methylhexaneamine be present in the extract of Geranium in an amount of at least 1% of the total mass of said extract.

As discussed previously, Geranium oil yields are generally in the range of less than 0.3% and methylhexaneamine represents less than 1% of the oil such that unadulterated extracts of Geranium contain less than 0.003% methylhexanamine, making production economically prohibitive. The methods and compositions of the present invention largely address this problem.

Methylhexanamine's safety profile is similar to caffeine. The LD50 for methylhexaneamine is 39 mg/kg for intravenous and 185 mg/kg for intraperitoneal administration (mouse), equivalent to 206 mg intravenously and 978 milligrams intraperitoneally injected for a 143 lb (65 kg) adult human. A typical dose for supplementation with the pure extract is 25-50 mg, or about 0.5 mg/kg of body weight. Caffeine's LD50 in mice is 62 mg/kg, and for an adult human female is 57 mg/kg, or 3.705 grams intravenously injected. Oral toxicity is much lower for caffeine (400-1,000 mg/kg).

While examining formulations containing basic extraction methods for Geranium, the inventors found that prior disclosed methods and materials were not very successful, based on methylhexaneamine content. As assayed by the inventors, it was further found that many commercially available Geranium-based products contained significantly lower methylhexaneamine levels than previously reported, and nearly all of those tested exhibited levels significantly below 1.0% methylhexaneamine. A single product reported to be an extract from Pelargonium hortorum exhibited 0.95% methylhexaneamine.

The inventors developed particular extracts of Geranium for use in pre-workout dietary supplements which exhibited methylhexaneamine levels consistently above 1.0%. The inventors further discovered that the extracts of the present invention exhibited the ability to suppress the appetite of a person. This is a surprising and previously unknown property that has not been demonstrated for extracts or oils of Geranium species or synthetic dimethylamylamine.

### SUMMARY OF THE INVENTION

Accordingly, the present invention provides methods and materials for making and using extracts of Geranium or Pelargonium comprising at least 1% methylhexaneamine. In certain embodiments, the present invention comprises extracts comprising at least 2% methylhexaneamine or at least 3% methylhexaneamine. In certain embodiments, the extract is made from natural sources, such as one or more plants or parts of plants. The plants may be of the genus Geranium or Pelargonium, preferably of the genus Geranium, and most preferably of the species Geranium robertianum or Geranium wilfordii.

In other embodiments, the present invention comprises extracts produced by:
a. providing a natural source of Geranium or Pelargonium, such as one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

In other embodiments, the invention comprises methods of processing Geranium or Pelargonium to provide an extract containing at least 1% methylhexaneamine, preferably at least 2% methylhexaneamine, and more preferably at least 3% methylhexaneamine. In certain embodiments, the method comprises:
a. providing a natural source of Geranium or Pelargonium, such as one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

In certain embodiments, the natural source is one or more plants from the genus Geranium sp.; preferably Geranium wilfordii Maxim or Geranium robertianum; and most preferably Geranium robertianum. In certain embodiments, drying the concentrated aqueous phase to obtain a powder is accomplished by spray drying. In certain embodiments, concentrating the aqueous phase is performed at low temperature. In certain embodiments, extracting the mixture to separate an essential oil phase from an aqueous phase is carried out for about three hours and repeated three times.

In certain embodiments, the extract produced comprises at least 1% methylhexaneamine, preferably at least 2% methylhexaneamine, and more preferably at least 3% methylhexaneamine.

In other embodiments, the present invention provides compositions comprising an extract of Geranium or Pelargonium, the extract comprising at least 1% methylhexaneamine, preferably at least 2% methylhexaneamine, and more preferably, at least 3% methylhexaneamine.

In certain embodiments, the composition comprises an extract which is produced by:
a. providing a natural source of Geranium or Pelargonium, such as one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

In certain embodiments, the natural source is Geranium sp.; preferably Geranium wilfordii Maxim or Geranium robertianum; and most preferably Geranium robertianum. In certain embodiments, the composition further comprises one or more additional ingredients selected from caffeine, citrulline and arginine. Suitable sources of caffeine include caffeine anhydrous, Coffea Arabica, Coffea canephora, Camellia sinensis, Ilex paraguariensis, Paullinia cupana, Theobroma cacao, and kola nut.

In other embodiments, the present invention provides compositions comprising an extract of Geranium or Pelargonium, the extract providing at least 1 mg methylhexaneamine per serving of the composition. In certain preferred embodiments, the extract provides at least 2 mg methylhexaneamine per serving of the composition. More preferably, the extract provides at least 3 mg methylhexaneamine per serving of the composition.

In yet other embodiments, the present invention provides methods for suppressing appetite in a subject, comprising administering a composition comprising an extract of Geranium or Pelargonium, wherein the extract contains at least 1% methylhexaneamine, preferably at least 2% methylhexaneamine, and more preferably at least 3% methylhexaneamine.

In certain embodiments, the extract of Geranium or Pelargonium is produced by:
a. providing a natural source of Geranium or Pelargonium, such as one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

In certain embodiments, the composition further comprises a source of caffeine. The source of caffeine may be selected from the group consisting of caffeine anhydrous, Coffea Arabica, Coffea canephora, Camellia sinensis, Ilex paraguariensis, Paullinia cupana, Theobroma cacao, and kola nut.

In certain embodiments, the natural source of Geranium or Pelargonium is a Geranium robertianum plant.

In certain embodiments, the method for suppressing appetite comprises administering a composition of the present invention, comprising at least 1 mg methylhexaneamine per serving, preferably at least 2 mg methylhexaneamine per serving and most preferably at least 3 mg methylhexaneamine per serving of the composition.

In other embodiments, the present invention comprises an appetite suppressing composition comprising an extract of Geranium or Pelargonium containing at least 1% methylhexaneamine. In certain embodiments, the appetite suppressing compostion contains at least 2% methylhexaneamine, preferably at least 3% methylhexaneamine.

In certain embodiments, the appetite suppressing composition of the present invention comprises an extract that contains at least 1% methylhexaneamine and is produced by:
a. providing a natural source of Geranium or Pelargonium, such as one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

In certain embodiments, the appetite suppressing composition of the invention further comprises a source of caffeine, which may be, for example, one or more of caffeine anhydrous, Coffea Arabica, Coffea canephora, Camellia sinensis, Ilex paraguariensis, Paullinia cupana, Theobroma cacao, or kola nut.

In certain embodiments, the appetite suppressing composition of the present invention is in the form of a meal replacement powder or beverage.

In certain embodiments, the present invention provides appetite suppressing compositions comprising at least 1 mg methylhexaneamine per serving of the composition; preferably at least 2 mg methylhexaneamine per serving of the composition; and more preferably, the composition comprises at least 3 mg methylhexaneamine per serving of the composition.

In certain embodiments, the composition further comprises a source of caffeine. Suitable sources of caffeine include one or more of the group consisting of: caffeine anhydrous, Coffea Arabica, Coffea canephora, Camellia sinensis, Ilex paraguariensis, Pauffinia cupana, Theobroma cacao, and kola nut.

The appetite suppressing compositions of the present invention may be in any physiologically acceptable form, and may be in the form of a meal replacement powder or beverage. The apppetite suppressing compositions of the present invention may further comprise one or more of the following additional ingredients: Hordenine, Rauwolfia; Beta-alanine; Citrulline; creatine [such as creatine HCl]; DMAE; Rhodiola extract; Inulin (Cichorium intybus) root; psyllium powder (Plantago ovata) seed husk; and oat straw powder (Avena sativa) leaf and stem; danol bitartrate; and vinpocetine.

In a first embodiment, the appetite suppressing composition of the present invention comprises: an extract of Geranium or Pelargonium, the extract comprising at least 1% methylhexaneamine, a source of caffeine, and at least one ingredient selected from the group consisting of: Hordenine, Rauwolfia, B-alanine, Citrulline, creatine, DMAE and Rhodiola extract.

In a second embodiment, the appetite suppressing composition of the present invention comprises: an extract of Geranium or Pelargonium, the extract comprising at least 1% methylhexaneamine, a source of caffeine, and at least one ingredient selected from the group consisting of: Inulin (Cichorium intybus) root, psyllium powder (Plantago ovata) seed husk, and oat straw powder (Avena sativa) leaf and stem.

In a third embodiment, the appetite suppressing composition of the present invention comprises: an extract of Geranium or Pelargonium, the extract comprising at least 1% methylhexaneamine, a source of caffeine, and at least one ingredient selected from the group consisting of: beta-alanine, citrulline, creatine HCl, Rhodiola extract, danol bitartrate, and vinpocetine

One embodiment of the present invention provides an extract of Geranium or Pelargonium from a natural source, said extract comprising at least 1% methylhexaneamine. Preferred natural sources include whole plants, which may be crushed, or parts thereof. In certain embodiments, the natural source is a Geranium sp. plant; preferably of the species Geranium wilfordii Maxim or Geranium robertianum; and most preferably of the species Geranium robertianum.

Another embodiment of the present invention provides methods of processing Geranium or Pelargonium to provide an extract containing at least 1% methylhexaneamine comprising:
a. Providing one or more crushed geranium plant;
b. Adding water and alcohol to the crushed geranium plant to obtain a mixture;
c. Reflowing the mixture;
d. Extracting the mixture to separate an essential oil phase from an aqueous phase;
e. Stewing and separating the essential oil phase to obtain a purified essential oil;
f. Concentrating the aqueous phase;
g. Drying the concentrated aqueous phase to obtain a powder; and
h. Combining the purified essential oil with the powder to obtain the extract.

Another embodiment of the invention provides a composition comprising an extract of Geranium or Pelargonium, the extract comprising at least 1% methylhexaneamine. In some aspects the extract of Geranium or Pelargonium comprises at least 2% methylhexaneamine. In other aspects the extract of Geranium or Pelargonium comprises at least 3% methylhexaneamine.

Another embodiment of the invention provides a composition comprising an extract of Geranium or Pelargonium, the extract providing at least 1 mg methylhexaneamine per serving of the composition. In some aspects the extract of Geranium or Pelargonium provides at least 2 mg methylhexaneamine per serving of the composition. In other aspects the extract of Geranium or Pelargonium provides at least 3 mg methylhexaneamine per serving of the composition.

Another embodiment of the invention provides a method of suppressing appetite in a subject, by use of a composition comprising an extract of Geranium or Pelargonium containing at least 1% methylhexaneamine.

Another embodiment of the present invention provides an appetite suppressing composition comprising an extract of Geranium or Pelargonium containing at least 1% methylhexaneamine.

Another embodiment of the present invention provides a method of suppressing appetite in a subject, by use of a composition comprising at least 1 mg methylhexaneamine per serving of the composition.

Another embodiment of the present invention provides an appetite suppressing composition comprising at least 1 mg methylhexaneamine per serving of the composition.

In preferred aspects of the invention, the Geranium or Pelargonium is Geranium sp.; preferably Geranium wilfordii Maxim or Geranium robertianum; and most preferably Geranium robertianum.

### BRIEF DESCRIPTION OF THE DRAWINGS

Embodiments will now be described, by way of example only, with reference to the attached Figure, wherein:

FIG. 1 is a bar graph showing the testing results of various Geranium oil samples and an extract according to an embodiment of the present invention.

### DETAILED DESCRIPTION

The present invention is directed toward methods of extraction, compositions and methods using such compositions comprising at least an extract of Geranium or Pelargonium from a natural source. According to various embodiments, the present invention is directed toward compositions and methods for suppressing appetite in a subject. More specifically, the present invention is directed towards a composition containing an effective amount of Pelargonium or Geranium species which include but are not limited to: Geranium maculatum, Geranium robertianum, Geranium wilfordii Maxim, Pelargonium crispum, Pelargonium cucullatum, Pelargonium echinatum, Pelargonium grandicalcaraturn, Pelargonium graveolens, Pelargonium magenteum, Pelargonium x nervosum, Pelargonium odorantissimum, Pelargonium peltatum, Pelargonium quercifolium, Pelargonium reniforme, Pelargonium sidoides, Pelargonium tomentosum, Pelargonium tricolor, Pelargonium xerophyton, Pelargonium zonale, Pelargonium grossalarioides, Pelargonium fragrans, Pelargonium capitatum, Pelargonium radens, Pelargonium citronellum, Pelargonium abrotanifolium, Pelargonium ionidiflorum, Pelargonium melissinum, Pelargonium nervosum, and Pelargonium x citrosum. The preferred species of Geranium or Pelargonium is Geranium sp.; preferably Geranium wilfordii Maxim or Geranium robertianum; most preferably Geranium robertianum.

The present invention provides improved methods of extraction from various Geranium species resulting in increased methylhexaneamine content. The present inventors attempted to improve upon the low yield of methylhexaneamine from previous methods of preparing an extract of Geranium from natural sources, such as whole Geranium plants, through hydro-alcoholic extraction with no added foreign ingredients. Such methods proved limited in that none of the various extraction methods explored using different species, yielded more than 0.5% methylhexaneamine.

The present inventors found that, utilizing the methods of the present invention, great improvements were achieved in the methylhexane-amine content in the Geranium extracts. Utilizing such methods, the present inventors identified the methods of the present invention, which resulted in methylhexaneamine content as follows: 0.4% in Geranium wilfordii Maxim and 0.9% in Geranium robertianum. The extraction method was further modified and the results were improved to 2.1% methylhexaneamine for Geranium wilfordii Maxim and 2.4% for Geranium robertianum. After further refinements, the extraction methods of the present invention achieved 2.7% methylhexaneamine for Geranium robertianum extract. Based on the results, it was found that Geranium robertianum extracted according to the method described herein achieved consistently greatly improved methylhexaneamine content. The inventors surprisingly discovered that an extract of Geranium prepared according to the preferred extraction procedure imparted a heretofore unobserved appetite suppressive effect.

The following extraction procedure was developed through various testing stages. Raw, whole plant material is washed, then crushed to a suitable size. The crushed plant material is mixed with an extraction solution of aqueous alcohol in suitable volume and reflowed. The mixture is extracted to collect the essential oil separate from the aqueous phase. The essential oil is further processed by stewing and separating to produce a purified oil; and the resulting aqueous extract is concentrated. The resulting aqueous concentrate is dried to a powder; and the powder is crushed, sifted and mixed with the purified essential oil to be prepared as a final extract. The preferred size for crushing the plant material is to 40 mesh. The preferred extraction solution is 50% ethanol in pure water. The extraction may be conducted from two to five hours and may be repeated. Preferably, extraction is carried out for three hours and repeated three times. Preferably, the aqueous extract is concentrated at low temperature. The preferred method of drying the aqueous concentrate is spray drying.

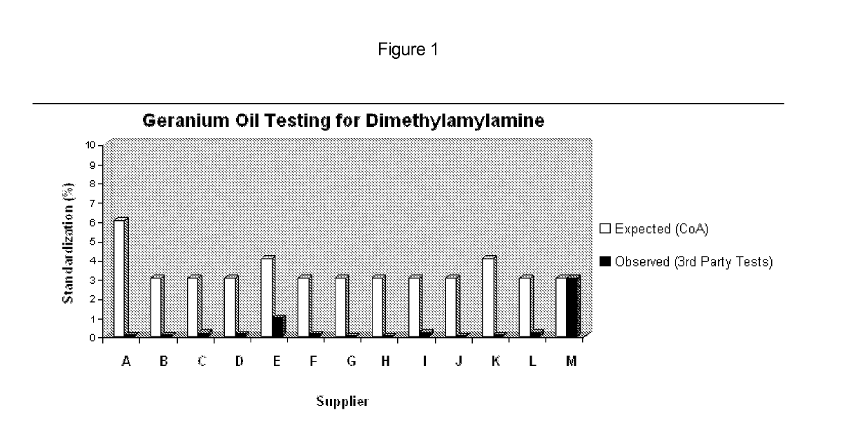

To further test the extract made according to an embodiment of the present invention, several commercially available Geranium oil samples were purchased for comparison to an extract of the present invention. The purchased samples were selected on the basis of reported methylhexaneamine content as per advertisements and certificates of analysis. Methylhexaneamine content of each sample was measured by high-performance liquid chromatography (HPLC). The results of the testing is shown in FIG. 1 where:
“A” represents a Pelargonium hortorum extract reported to be 6% methylhexaneamine; Expected: 6%; Measured: <0.05% (w/w).
“B” represents a Pelargonium hortorum extract reported to be 3% methylhexaneamine; Expected: 3%; Measured: <0.05% (w/w).
“C” represents a “Geranium oil” sample of undeclared species; Expected: 3%; Measured: 0.16% (w/w).
“D” represents a Pelargonium graveolens extract; Expected: 3%; Measured: <0.10% (w/w).
“E” represents a Pelargonium hortorum extract reported to be 130:1; Expected: 4%; Measured: 0.95% (w/w).
“F” represents a Pelargonium graveolens extract; Expected: 3%; Measured: <0.1% (w/w).
“G” represents a Pelargonium graveolens extract reported to be 200:1; Expected: 3%; Measured: 0.027% (w/w).
“H” represents a Pelargonium graveolens extract reported to be 100:1; Expected: 3%; Measured: 0.025% (w/w).
“I” represents a “Geranium oil” sample of undeclared species; Expected: 3%; Measured: 0.20% (w/w).
“J” represents a Pelargonium graveolens extract reported to be 100:1; Expected: 3%; Measured: <0.005% (w/w).
“K” represents a Pelargonium graveolens extract reported to be 200:1; Expected: 4%; Measured: <0.05% (w/w).
“L” represents a Pelargonium graveolens extract (stem and root) reported to be 5:1; Expected: 3%; Measured: 0.19% (w/w).
“M” represents a Geranium robertianum extract in accordance with an embodiment of the present invention; Expected: 3%; Measured: 3.06% (w/w).

It should be noted that, with the exception of the embodiment of the present invention, all of the above ‘geranium oil’ samples are actually extracted from the ‘scented geranium’ or storksbillls (Pelargonium sp.), or of undeclared species, rather than being identifiable as extracts from the ‘hardy geranium’ of the genus Geranium, or cranesbills. Although at one time, Geranium and Pelargonium were both included in a common family, Geraniaceae, along with the genus Erodium. (also referred to as filarees or heronsbills). However, the three are sufficiently distinct that they are now classified as separate genera. Accordingly, it is not believed that any of the commercially available ‘geranium oil’ extracts are extracted from species of the genus Geranium. Shown is the expected content of methylhexaneamine, as well as the measured amount. HPLC was calibrated with three known amounts of synthetic methylhexaneamine to span the expected content of the samples. Surprisingly, as can be seen, only sample “M” (the inventive sample) contained the expected amount of methylhexaneamine. None of the other samples tested exhibited greater than 0.95% methylhexaneamine, and most exhibited significantly lower levels of methylhexaneamine.

The inventors separately tested 16 additional species of Pelargonium (P. crispum—Cinnamon Germanium, P. denticulatum—Ferleaf Germanium, P. quercifolium—Almond Germanium, P. radens—Skeleton-Rose Germanium, P. tomentosum—Peppermint Germanium, P. graveolens—Robert's Lemon-Rose Germanium, P. grossularioldes—Coconut Germanium, P. odoratissimum—Apple Germanium, Pelargonium “Bontrosai”—Lemon Sculpture Germanium, P. citronellum—Mabel Grey Germanium, P. quercifolium—Chocolate Mint Germanium, P. pellatum—Austrian Germanium, P. xnervosum—Lime Germanium, P. capitatum—Attar of Rose Germanium, P. scabrum—Apricot Germanium, and P. citrosum—Prince of Orange Germanium) as suitable sources of starting material from which to obtain natural methylhexaneamine. All samples were first tested as 50:50 aqueous alcohol mixture (methanol) of dried leaves. Samples were tested for methylhexaneamine by HPLC. Of the 16 sample species, only one (Pelargonium citronellum) resulted in detection of methylhexaneamine at the detection threshold. These data suggest that Pelargonium citronellum is a particularly good source of methylhexaneamine, particularly when processed according to the method herein described.

The inventors undertook a series of observational studies to assess the effectiveness of acute administration of compositions according to various embodiments of the present invention.

### EXAMPLE 1

Samples in accordance with the an aspect of the present invention were formulated as follows:
Dose per pill (3 pills total serving size):
129 mg Caffeine and
3 mg methylhexaneamine (from Geranium robertianium extract).

Geranium was in the form of a Geranium robertianium extract prepared according to the preferred extraction method herein previously described.

### EXAMPLE 2

Samples according to an embodiment of the present invention were formulated as follows:
Dose per pill (3 pills total serving size):
129 mg Caffeine,
3 mg methylhexaneamine (from Geranium robertianium extract),
16.67 mg Hordenine, and
1.3 mg Rauwolfia.

The formulation of Example 2 was as in Example 1 with the addition of 1.3 mg Rauwolfia in the form of Rauwolfia vomitoria (Root Bark, ethanol extract) and Hordenine.

### EXAMPLE 3

Samples according to an embodiment of the present invention were formulated as follows:
Dose per pill (3 pills total serving size):
200 mg Caffeine,
13.5 mg methylhexaneamine (from Geranium robertianium extract).

### EXAMPLE 4

Samples according to an embodiment of the present invention were formulated as follows:
Dose per pill (3 pills total serving size):
200 mg Caffeine,
16 mg methylhexaneamine (from Geranium robertianium extract).

### EXAMPLE 5

A pre-exercise supplement according to an embodiment of the present invention was formulated as follows, as a powder for administration prior to exercise and contained the following ingredients with 1 to 3 servings to be consumed at the discretion of the subject:
Dose per scoop/serving:
120 mg Caffeine,
5.3 mg methylhexaneamine (from Geranium robertianium extract),
1 g B-alanine,
1 g Citrulline, and
1 g creatine.

### EXAMPLE 6

A pre-exercise supplement according to an embodiment of the present invention was formulated as follows, as a powder for administration prior to exercise and contained the following ingredients with 1 to 3 servings to be consumed at the discretion of the subject:
Dose per scoop/serving:
120 mg Caffeine,
5.3 mg methylhexaneamine (from Geranium robertianium extract),
1 g B-alanine,
1 g Citrulline,
1 g creatine,
6 mg DMAE, and
6 mg Rhodiola extract.

All samples from Examples 1 through 6 were given to groups of subjects on separate occasions to assess effects and subject satisfaction; also, some samples were given multiple times on separate occasions. The pool of subjects consisted of 32 individuals (29 male, 3 female) given various samples in groups of at least 10. All subjects were healthy between the ages of 20 and 35 years; all were at least moderately active; and most (>70% in any one assessment) were regular consumers of stimulants. Regular use of stimulants was considered to be at least daily consumption of caffeinated beverages but most subjects also reported as being regular consumers of pre-workout energy supplements. Pre-workout energy supplements are consumed primarily prior to an exercise session to increase physical energy and mental focus/awareness to enhance the exercise.

Subjects were instructed to maintain normal diet and activity. Subjects were given the samples, encouraged to use the sample in-place of any regular pre-workout product prior to the next exercise session, and subsequently surveyed for ratings of perceived effects on energy levels and mental focus. Subjects were also advised to supply general comments regarding the supplement in terms of negative side effects, comparison to other products, overall satisfaction and effectiveness.

Overall, a majority of subjects were satisfied with the test samples in terms of energy and focus and preferred them to any regularly consumed products, most of which would contain comparable amounts of caffeine and additional ingredients and none of which would have contained Geranium or Pelargonium extract according to the present invention. This is particularly surprising when considering that the majority of subjects were regular caffeine users and that the sample formulations were taken in place of any normally consumed pre-workout supplement. The inventors determined that this is due to the extract according to the invention, rather than the caffeine.

An unexpected and surprising result of the instant invention was that, while the formulations according to embodiments of the present invention were being examined primarily for increasing energy and focus, many individuals, despite not being questioned on appetite, reported effects of reduced appetite. None of these reports was associated with any negative effects. As noted, since most subjects were regular consumers of caffeine, the inventors determined this surprising effect on appetite was due to the extract made in accordance with the present invention. To the inventors' knowledge, this surprising effect has not been previously attributed to and Geranium or Pelargonium extract or to dimethylamylamine.

Subsequently, three subjects were administered 156 mg of Geranium robertianium extract (providing 3 mg methylhexaneamine) made in accordance with the preferred extraction method. All three subjects reported reduced appetite which lasted for several hours.

The amount of Pelargonium or Geranium extract may be, preferably from about 40 mg to about 600 mg per serving of the composition.

More preferably, the amount of Pelargonium or Geranium extract may be from about 100 mg to about 300 mg per serving of the composition. Preferred embodiments of the present invention provide Pelargonium or Geranium extract supplying from about 1 mg to about 20 mg methylhexaneamine per serving of the composition. More preferably, the present invention provides Pelargonium or Geranium extract supplying from about 10 mg to about 16 mg methylhexaneamine per serving of the composition.

Additional embodiments of the present invention provide for compositions comprising from about 1 mg to about 20 mg methylhexaneamine per serving of the composition. More preferably, the present invention, in certain aspects, provide for compositions comprising from about 10 mg to about 16 mg methylhexaneamine per serving of the composition.

Additional embodiments of the present invention provide for appetite suppressing compositions comprising from about 1 mg to about 20 mg methylhexaneamine per serving of the composition. More preferably, the present invention, provides for appetite suppressing compositions comprising from about 3 mg to about 18 mg methylhexaneamine; 5 mg to about 16 mg methylhexaneamine; or 10 mg to about 16 mg methylhexaneamine per serving of the composition.

The compositions, in accordance with various embodiments of the present invention, may by provided for administration in any suitable form commonly known in the art. Such forms include but are not limited to: pills, capsules, tablets, caplets, bars, powders, and ready-to-drink beverages.

According to some aspects, the compositions are provided as meal replacement dietary supplements. Such meal replacement dietary supplements are intended to be consumed in-place of some of an individual's meals and prevent intake of excess, unwanted calories. As such, meal replacement dietary supplements in accordance with aspects of the present invention may be flavored to be pleasant tasting. They may also contain additional ingredients such as fiber and protein to promote satiety and fullness, and vitamins and minerals to supplement the diet of the individual.

Suitable fibers include both soluble and insoluble dietary fibers. Examples of suitable fibers include flax seed, oat bran, pea fiber, bamboo fiber, chia seed, psyllium seed husk, glucomannan, chitosan, inulin, pectin, polydextrose, gum arabic, guar gum, and digestion resistant maltodextrin.

### EXAMPLE 7

Appetite Suppression Dietary Supplement. The following high-fiber supplement to reduce appetite is prepared as a powder to be mixed with water and consumed as a beverage twice daily. One serving (about 8 grams) contains:
6 g Inulin (Cichorium intybus) root,
150 mg caffeine anhydrous,
10 mg psyllium powder (Plantago ovata) seed husk,
10 mg oat straw powder (Avena sativa) leaf and stem,
5.3 mg methylhexaneamine (from Geranium robertianum extract), excipients and flavorings.

### EXAMPLE 8

Appetite Suppression Dietary Supplement. The following supplement to reduce appetite is prepared as a capsule to be taken prior to meal consumption. One serving contains:
450 mg Geranium extract (providing 13.5 mg methylhexaneamine) and excipients.

### EXAMPLE 9

Appetite Suppression Dietary Supplement. The following supplement to reduce appetite is prepared as a capsule to be taken prior to meal consumption. One serving contains:
500 mg Geranium extract (providing 10 mg methylhexaneamine),
100 mg caffeine anhydrous, and excipients.

### EXAMPLE 10

Pre-workout Dietary Supplement. One to three servings of following formula prepared as a powder is to be taken by an individual 5 to 30 minutes prior to exercise. One serving of the formula contains:
120 mg caffeine anhydrous,
5.3 mg methylhexaneamine (from Geranium robertianum extract),
1 g beta-alanine,
1 g citrulline,
1 g creatine monohydrate,
excipients and flavorings.

### EXAMPLE 11

Appetited Suppression Dietary Supplement. As a powder, 1 serving is about 5 g. 1-3 servings mixed with cold water to be taken 30-45 minutes before exercise. One serving of the formula contains:
1 g beta-alanine
1 g citrulline
1 g creatine HCl
177 mg Geranium robertianum extract (supplying 5.3 mg methylhexaneamine)
120 mg caffeine anhydrous
17 mg Rhodiola extract
17 mg danol bitartrate.
1.67 mg vinpocetine.

### EXAMPLE 12

A pre-exercise supplement according to an embodiment of the present invention was formulated as follows, as a powder for administration prior to exercise and contained the following ingredients with 1 to 3 servings to be consumed at the discretion of the subject:
Dose per scoop/serving:
120 mg Caffeine,
5.3 mg methylhexaneamine (from P. citronellum extract),
1 g B-alanine,
1 g Citrulline, and
1 g creatine.

### EXAMPLE 13

Appetite Suppression Dietary Supplement. The following high-fiber supplement to reduce appetite is prepared as a powder to be mixed with water and consumed as a beverage twice daily. One serving (about 8 grams) contains:
6 g Inulin (Cichorium intybus) root,
150 mg caffeine anhydrous,
10 mg psyllium powder (Plantago ovata) seed husk,
10 mg oat straw powder (Avena sativa) leaf and stem,
5.3 mg methylhexaneamine (from P. citronellum extract), excipients and flavorings.

The above-described embodiments are non-limiting examples of the present invention. As will be appreciated by those of skill in the art, numerous alterations and modifications may be effected thereto without departing from the scope of the invention, which is defined solely by the claims appended hereto.

1. An extract of Pelargonium isolated from natural sources, said extract comprising at least 1% methylhexaneamine.

2. The extract of claim 1 wherein the extract is produced by:
a. providing one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

3. A method of processing Pelargonium to provide an extract containing at least 1% methylhexaneamine comprising:
a. providing one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

4. The method of claim 3 wherein drying the concentrated aqueous phase to obtain a powder is accomplished by spray drying.

5. The method of claim 3 wherein concentrating the aqueous phase is preformed at low temperature.

6. The method of claim 3 wherein extracting the mixture to separate an essential oil phase from an aqueous phase is carried out for about three hours and repeated three times.

7. A composition comprising the extract of claim 1.

8. The composition of claim 7 wherein the extract is produced by:
a. providing one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

9. The composition of claim 7 further comprising at least one of the following additional ingredients: caffeine, citrulline and arginine.

10. The composition of claim 7, A composition comprising an extract of Pelargonium citronellum, the extract providing at least 1 mg methylhexaneamine per serving of the composition.

11. The composition of claim 10 wherein the extract is produced by:
a. providing one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

12. The composition of claim 11 further comprising caffeine.

13. A method for suppressing appetite in a subject, comprising administering the composition of claim 7.

14. The method of claim 13 wherein the composition further comprises a source of caffeine, selected from the group consisting of caffeine anhydrous, Coffea Arabica, Coffea canephora, Camellia sinensis, Ilex paraguariensis, Paullinia cupana, Theobroma cacao, and kola nut.

15. An appetite suppressing composition comprising the extract of claim 1.

16. The appetite suppressing composition of claim 15 wherein the extract is produced by:
a. providing one or more whole crushed geranium plants;
b. adding water and alcohol to the crushed plant to obtain a mixture;
c. reflowing the mixture;
d. extracting the mixture to separate an essential oil phase from an aqueous phase;
e. stewing and separating the essential oil phase to obtain a purified essential oil;
f. concentrating the aqueous phase;
g. drying the concentrated aqueous phase to obtain a powder; and
h. combining the purified essential oil with the powder to obtain the extract.

17. The appetite suppressing composition of claim 15 further comprising a source of caffeine, selected from the group consisting of caffeine anhydrous, Coffea Arabica, Coffea canephora, Camellia sinensis, Ilex paraguariensis, Paullinia cupana, Theobroma cacao, and kola nut.

18. The appetite suppressing compostion of claim 15 in the form of a meal replacement powder or beverage.

19. An appetite suppressing composition comprising at least 1 mg methylhexaneamine per serving of the composition.

20. The composition of claim 10 further comprising a source of caffeine selected from the group consisting of caffeine anhydrous, Coffea Arabica, Coffea canephora, Camellia sinensis, Ilex paraguariensis, Paullinia cupana, Theobroma cacao, and kola nut.

21. The apppetite suppressing composition of claim 20, further comprising at least one ingredient selected from the group consisting of: Hordenine, Rauwolfia; B-alanine, Citrulline, creatine, DMAE and Rhodiola extract.

22. The appetite suppressing composition of claim 20, further comprising at least one ingredient selcted from the group consisting of: Inulin (Cichorium intybus) root, psyllium powder (Plantago ovata) seed husk, and oat straw powder (Avena sativa) leaf and stem.

23. The extract of claim 1, wherein the extract is isolated from Pelargonium citronellum.
